(19) United States
(12) Patent Application Publication
(10) Pub. No.: US 2016/0296375 A1
(43) Pub. Date: Oct. 13, 2016

(54) SYSTEM AND METHOD FOR PRODUCING ASSISTANCE INFORMATION FOR LASER-ASSISTED CATARACT OPERATION

(71) Applicant: CARL ZEISS MEDITEC AG, Jena (DE)

(72) Inventors: Matthias REICH, Jena (DE); Michael Stefan RILL, Jena (DE)

(21) Appl. No.: 15/036,767

(22) PCT Filed: Nov. 11, 2014
(86) PCT No.: PCT/EP2014/074290

§ 371 Date: May 13, 2016

(30) Foreign Application Priority Data
Nov. 13, 2013 (DE) 10 2013 223 152.0

(51) Int. Cl. A61F 9/008; A61B 3/13

(52) U.S. Cl. CPC A61F 9/00825; A61B 3/13; A61F 2009/0087; A61F 2009/00872; A61F 2009/00887

(57) ABSTRACT

A system for producing assistance information for a laser-assisted cataract operation on an eye, including: a laser cataract device with a laser beam source, a laser beam scanning device, a control device for predefining a cutting geometry of the cuts, an imaging device and an output interface. The control device determines the position of reference structures of the eye and produces a position data set containing cutting geometry in relation to the structures, and outputs the position data record at the interface. The system also includes an operation microscope having a microscope, a display device, an input interface for receiving the data set, and a control unit that receives the data set and determines the position of the structures with respect to image data, and inputs the cutting geometry into the image displayed on the display device appropriately in terms of position and size with respect to the reference structures.

### PRIORITY CLAIM

The present application is a National Phase entry of PCT Application No. PCT/EP2014/074290, filed Nov. 11, 2014, which claims priority from German Patent Application Number 102013223152.0, filed Nov. 13, 2013, the disclosures of which are hereby incorporated by reference herein in their entirety.

### FIELD OF THE INVENTION

The invention relates to a system and a method for producing assistance information for the laser-assisted cataract operation.

### BACKGROUND OF THE INVENTION

Cataract operations are assumed to be the most frequently carried out surgical intervention on the eye (cf. S. Bali et al., “Early experience with the femtosecond laser for cataract surgery”, Ophthalmology, May 2012, 119 (5), pages 891-899). Approximately one-third of the population of the developed world undergoes this operation (cf. Palanker et al., “Femtosecond laser-assisted cataract surgery with integrated optical coherence tomography”, Sci. Transl. Med 2010 (2): 58RA85; available at www.stanford.edu/˜palanker/publications/fs_laser_cataract.pdf). The requirements for the efficiency and standard of quality with which this operation is carried out are correspondingly high.

During cataract operation, incisions are made in the cornea, through which a surgical instrument can be introduced into the eye. Furthermore, the capsular bag is opened with a surgical cut in order to remove the lens located therein and to be able to insert an intraocular lens (IOL). While, in the past, these cuts were performed mechanically, devices are now known with which one cut or both cuts can be produced by laser radiation (cf. named publications by Bali et al., Palanker et al. and J. Talamo et al., “Optical patient interface in femtosecond laser-assisted cataract surgery: contact corneal applanation versus liquid immersion”, J. Cataract Surg., Vol. 39, April 2013, pages 501-510). An fs laser cataract device is also known from WO 2009/039302 A2. This is a laser-based device which produces cuts in the eye by means of laser radiation. The laser radiation is usually moved according to a predetermined path, wherein the path fills the surface of the cut to be produced. The movement of the laser beam is effected by means of a laser beam scanning device which, together with the laser beam source providing the laser beam, is controlled by a control device. The latter predefines a cutting geometry of the cuts by controlling the laser beam source and the laser beam scanning device .according to control data. As a result, the control data thus define the cut surface to be produced and the laser cataract devices can thus produce very fine cuts extremely precisely.

The cuts to be produced are opening cuts in the cornea which enable access of instruments into the anterior chamber of the eye. Through this opening, the surgical instruments are introduced into the eye during the subsequent operation. A further cut opens the capsular bag. It effects the so-called anterior capsulotomy. Ideally, the capsular bag is opened with a circular cut. After removal of the part of the capsular bag exposed and isolated by the cut, the lens of the eye is comminuted and removed. Both tearing of the capsular bag and equally an inadvertent severing of the posterior wall of the capsular bag represent an undesired complication of the operation which is to be avoided as much as possible (see Bali et al.).

Equally disruptive is an incomplete cut which does not effect a continuous cutting line but leaves behind incompletely severed bridges of material (see Talamo et al.), since here there is the risk of undesired tearing of the capsular bag when the lens is grasped in the capsular bag.

In the case of the use of laser cataract devices, the cataract operation is usually carried out in two stages (see Bali et al.). First, the cut surfaces are produced; at least one with the laser cataract device. Then the patient is brought under an operation microscope so that the surgeon sees the eye to be treated enlarged when he removes the lens of the eye through the cuts produced and inserts the IOL. Assistance devices for operation microscopes are known which superimpose information for the surgeon, over the eye to be treated. Such an assistance device is offered, for example, by Carl Zeiss Meditec AG under the name CALLISTO Eye. It displays both planned cuts on the cornea and the capsular bag and also markings for correct alignment of the eye and can adapt these to eye movements in real time. Such display is also known for laser cataract devices, for example from DE 102011082901 A1.

### SUMMARY OF THE INVENTION

Therefore, both laser cataract devices and operation microscopes are known in the state of the art, which provide assistance functions superimposing the position of planned cuts on a display of the eye. The object of the invention is to further assist a surgeon during the laser-assisted cataract operation with the result that the quality and also the efficiency of the cataract operation is increased.

This object is achieved according to the invention with a system for producing assistance information for a laser-assisted cataract operation on an eye, wherein the system comprises:

a laser cataract device, which comprises
a laser beam source which emits a laser beam, and a laser beam scanning device for producing cuts in the cornea and/or capsular bag of the eye by scanning the laser beam in the cornea and/or capsular bag of the eye,
a control device for predefining a cutting geometry of the cuts by control to control the laser beam source and the laser beam scanning device,
an imaging device for imaging reference structures of the eye,
an output interface,
wherein the control device is designed to determine the position of the reference structures and to produce a position data set which contains the cutting geometry and the relative position thereof in relation to the reference structures and to output it on the interface,

an operation microscope, which comprises
a microscope and/or a camera for imaging the eye on an image sensor which supplies image data,
a display device for displaying an image of the eye according to the image data,
an input interface for receiving the position data set and
a control unit which receives the position data set on the input interface, determines the position of the reference structures of the eye with respect to the image data and, on the display device, superimposes the cutting geometry contained in the position data set on the displayed image appropriately in terms of position and size with respect to the reference structures.

The object is further achieved according to the invention with a method for producing assistance information for a laser-assisted cataract operation which, produces cuts in the cornea and/or capsular bag of the eye by a laser cataract device, controlled according to control data, wherein in the method the following steps are performed:
a) imaging the eye and determining a position of reference structures of the eye and determining a cutting geometry of the cuts produced or to be produced from the control data,
b) determining a relative position of the cutting geometry in relation to the reference structures and providing a position data set which contains the cutting geometry and the relative position thereof in relation to the reference structures,
c) transferring the position data set to an operation microscope,
d) producing and displaying an image of the eye with the operation microscope,
e) determining the position of the reference structures of the eye in the image produced and
f) with respect to the reference structures, superimposing on the displayed image the cutting geometry contained in the position data set appropriately in terms of position and size.

The invention provides a system consisting of a laser cataract device and an operation microscope. The laser cataract device provides the position data set and in addition determines the position of the cutting geometry with respect to the position of the reference structures. The cutting geometry results from the control data with great accuracy. The control device determines the position of the reference structures and registers the cutting geometry either directly on these reference structures or registers the position of the reference structures and the cutting geometry on a common reference system, e.g. a coordinate system. This can be the coordinate system which is used in the laser cataract device, e.g. the laser beam scanning device. The operation microscope belonging to the system imports the position data set. It determines the position of the reference structures, e.g. in the image data of the enlarged imaging of the eye which the microscope produces. As the position data set made available to the operation microscope reflects the relative position of the reference structures and the cutting geometry, the control unit can superimpose the cutting geometry on the displayed image appropriately in terms of position and size.

As the cutting geometry is known extremely accurately on the basis of the control data, which ultimately is also due to the fact that the laser cataract device has produced or will yet produce a cutting geometry corresponding to the control data which is reproducible with a high degree of accuracy, the operation microscope no longer displays, as in the state of the art, a target for cut surfaces but the cuts which were or are ultimately actually produced. These can be cuts in the capsular bag, lens and/or cornea. The surgeon can e.g. see the cuts produced by the laser cataract device.

The system according to the invention and the device according to the invention allow the laser cataract device to be operated in such a way that a particularly fine cut surface is produced, e.g. a cut surface which a surgeon would not be able to identify in the operation microscope, or only with difficulty. The fineness of a cut surface in the case of a laser cataract device depends on laser parameters. Usually, with pulsed laser radiation, a sequence of optical break-throughs are produced and each optical break-through produces cavitation. Immediately after the production of the cut, the cut surface is visible in the form of a carpet of the induced cavitations. Depending on the laser beam parameters, these bubbles disappear more or less quickly and more or less completely. The invention now allows working with cavitations which disappear quickly and/or completely. In this case, a surgeon would have problems identifying the cuts produced under the operation microscope. In an embodiment of the invention, the laser cataract device uses a cut surface production which even works without optical break-throughs. Such cut surface productions are known in the state of the art and produce particularly smooth cuts. Unsurprisingly, smooth cuts in the cornea and in the capsular bag are advantageous for the optical result of the cataract operation.

In a perfectly carried out capsulotomy cut, a circular capsular bag segment is completely detached. However, this state is not always achieved. Sometimes, folds in the cornea cause the focal positioning of the laser cataract device not to lie exactly in the capsule wall or the intensity at the focal point is too low for an optical perforation with the result that the severing of the capsule wall is incomplete at some points. In a preferred further development of the invention, the control device detects any gaps in cuts by means of the imaging device. By gaps is meant incompletely severed areas which lie at locations at which the control data predefine a cut. The position of such gaps is recorded in the position data set. With regard to the registration, what was said for the cutting geometry also applies to the position of the gaps. The control unit of the operation microscope superimposes the gaps on the displayed image. In this way, in the case of gaps, i.e. incompletely severed points, the surgeon can pay particular attention to the manual removal of the capsular bag segment which was isolated through the capsulotomy cut. The risk of tearing the capsular bag and thus causing a complication in the operation can be avoided because, by means of the further development, the surgeon receives information about possible incomplete capsulotomy cuts and the problem points resulting therefrom are displayed visually in the microscope.

In the operation microscope, the superimposed cutting geometry and further data on eye movements are preferably tracked. This can be referred to as real-time registration.

The position of the cut or the cuts which the laser cataract device performs automatically is determined for the laser beam scanning device by the control data. The control data usually relate to the reference system of the laser cataract device. The cutting geometry can, in an embodiment which is particularly easy to realize, be saved in the position data set in the form of the control data and thus form a basis for the information to be transferred to the operation microscope.

Among other things, the edge positions of the pupil of the eye and/or limbus positions come into consideration as reference structures. Vessel and iris structures of the eye can also be used as reference structures. They can be determined as reference structures. If the position thereof with respect to the reference system of the laser cataract device is indicated, it is sufficient to indicate the position of the reference structures in this reference system and to save it in the position data set. Thus, both the cutting geometry and the reference structures in respect of their position are related to a common reference system, e.g. that of the laser beam scanning device.

The position detection is effected by means of the imaging device. This can be a navigation system which is contained in the laser cataract device and makes it easier for the surgeon to align the eye correctly to the laser cataract device (and thus ultimately to the cutting geometries which the laser cataract device provides and produces). The imaging device can operate on the basis of optical coherence tomography but other methods, such as Scheimpflug photography, confocal detection or detection in a video system, are also possible. It is only important that the position of the imaging device in relation to the laser beam scanning device or to the reference system thereof is known.

In order to be able to make a precise description of the cutting position in relation to the reference system of the eye, the angle of rotation of the eye about the optical axis is preferably determined in relation to the coordinate system of the laser beam scanning device of the laser cataract device. In the simplest case, the angle arrangement can be regarded as being predefined by the alignment of the head with respect to the laser cataract device. If a more precise indication is desired, it is possible to analyse iris structures and/or scleral vessel structures near the limbus with the imaging device and to indicate their position with respect to the laser beam scanning device.

If a patient interface in use does not make it possible to see the named structures of the eye, the angle arrangement is preferably determined with reference to the named structures immediately before or during the docking of the patient interface on the eye.

In most cases, the patient interface has two connection points: first of all, the patient interface is connected to the sclera of the eye by a vacuum. It is then connected firmly to the laser cataract device by means of suitable mechanical interfaces. In the case of such a bilaterally connected patient interface, in an embodiment of the invention it is provided to implement a measurement system in the laser cataract device which captures an angle of rotation at the mechanical interface between contact element and laser cataract device when the patient interface was attached to the laser cataract device. In a preferred embodiment, the measurement system comprises an optical scale or marking which is applied to the patient interface and can be read or identified with respect to its position both with the imaging device of the laser cataract device during the docking of the patient interface on the eye and when the patient interface is fixed to the laser cataract device. Optional and alternative methods comprise electrical or magnetic measurement methods.

The action of a patient interface can also be used for the reference structure. Then it is determined at which position and in which orientation the patient interface was docked on the patient's eye. In addition, in a further development or independent invention, the fact is exploited that ridges which came to lie on the eye through the suction of the patient interface (for example marking projections or indentations or also a suction edge in the case of a vacuum fixing) leave visible marks on the cornea or conjunctiva. Pressure points on the epithelium or small bleeds in the conjunctiva are produced deliberately. It is therefore provided to design the pressure profile of a patient interface in such a way that it makes it possible to identify a positional orientation, e.g. a clear axis orientation of the eye, for example by means of an asymmetrical marking (e.g. in the form of an interrupted edge structure) or by means of separate pressure markings which clearly indicate the rotational position of the eye. The eye with the pressure marks, which serve as reference structures, is then imaged (e.g. with the imaging device of the laser cataract device). On the basis of this information, the position is determined which the eye had on the device which used the patient interface. In the case of the system, the laser cataract device produces the position data set. The coordinates of the reference systems of the laser cataract device and of the operation microscope can then be aligned with each other such that the cutting geometry is superimposed on the displayed image of the operation microscope appropriately in terms of position and size. In a further development of this marking principle, specific illumination is effected which highlights the pressure marks in the imaging. For example, glancing illumination can be used in order to clearly highlight the pressure marks.

As examinations have shown (see Talamo et al.), gaps in cuts can originate from folds in the cornea. In a further development it is therefore preferred to detect folds in the cornea directly with the imaging device, to determine the position thereof and to record the positions of these gaps in the position data set. The locations of these folds can then, as for the locations of the previously discussed gaps, be superimposed on the displayed image. At the points at which a corneal fold crosses a cutting path, the cut surface can be incomplete. It is therefore particularly preferred to superimpose the crossing points between cutting geometry and locations of the corneal folds on the displayed image and particularly preferred to mark them. They are potential locations for an incomplete cut surface. A surgeon can thereby take them into account and avoid an undesired tearing of the capsular bag. Under suitable illumination conditions, the folds can be identified in a video image in the form of linear signals. The detection of corneal folds present can be effected with a navigation system present elsewhere in the laser cataract device. Corneal folds also become apparent in an OCT image, for example through bulges in the posterior surface of the cornea.

The position data set determined in the laser cataract device is preferably made available to the operation microscope via a data export module. The data transfer can be effected electronically (e.g. via wired or wireless networks), by means of a storage medium (e.g. USB stick) or by means of plain radio transmission (e.g. Bluetooth). The transfer is preferably encrypted. In a preferred embodiment, the operation microscope has a data import module which imports the position data set from the laser cataract device.

The operation microscope determines the position of the reference structures. This is preferably effected in the image data which are produced in the enlarged imaging of the eye. Alternatively, a separate camera device can also be used which supplies an additional imaging of the eye for the determination of the position of the reference structures. In the variant which produces pressure marks, an additional imaging is advantageous. In the operation microscope, the scaling and alignment of the superimposed cutting geometry is effected, for example, with reference to edges of the pupils and/or limbus, on the orientation and scale of the image of the eye which the microscope supplies. A corresponding rotation can be carried out with reference to iris and/or vessel structures which are used as reference structures. Alternatively, the rotation can also be derived from a known position of the cut surfaces with respect to the eye. The eye is then adjusted in the laser cataract device into a particular rotational position and the operation microscope identifies a deviating position of the eye with reference to iris and/or vessel structures.

Within the framework of the cataract operation, in some embodiments, a fragmentation of the lens of the eye (lens fragmentation) is also performed with the laser cataract device, in which the lens of the eye is fragmented by means of fragmentation cuts. It is intended that the posterior capsular bag and the iris remain undamaged. Safety zones are therefore provided in relation to the posterior capsular bag and the iris in which no fragmentation cuts are effected. These areas can preferably be identified by the control device by means of the imaging device and recorded in the position data set. Often, despite the application of the laser pulses, no cutting effect takes place in the posterior area of the lens because the scattering in the area of the lens penetrated by radiation weakens the laser energy too much and/or enlarges the focal point too much. This can also be identified by the control device by means of the imaging device, for example, through a lack of cavitation formation on the production of cuts by means of optical perforations which produce cavitations. The control unit can superimpose on the displayed image of the operation microscope the areas at which there has/has not been fragmentation of the lens. Depending on the technique used to remove the lens, for example when phacoemulsification is used, it can be advantageous to perform fragmentation cuts in some areas of the lens more finely. This information is also preferably superimposed on the image of the operation microscope. In particular surgical techniques it is desired to perform a posterior capsulotomy in addition to an anterior capsulotomy. This cutting geometry is also preferably recorded in the position data set and displayed in the operation microscope.

Particular attention must be paid in the case of a cataract operation to those patients on whom a refractive operation for defective vision has already been carried out. In the case of patients with previous refractive laser surgery (e.g. LASIK), the knowledge of cut edges in the case of the production of the lamellae, which had exposed the inside of the cornea, are of interest for example. These edges can additionally be identified with the imaging device of the laser cataract device, recorded in the position data set and represented visually in the operation microscope. A surgeon can thereby better take them into account.

The method according to the invention produces assistance information in the framework of a cataract operation. The method does not require that surgical steps are performed. The steps of imaging and determining the relative position can be performed both before and after the production of the cut surfaces. This is because the control data, which are used in the production of the cut surfaces in the eye, indicate the position of the cutting geometry precisely. The method is therefore not directed at measuring produced cut surfaces. It does not require a surgical step. The same applies to the steps which are performed by the method on the operation microscope. Here too, no surgical intervention is necessary. The method claim is therefore also directed at the production of information within the framework of a cataract operation. This information makes the intervention easier for the surgeon. The method for producing these data is not connected to a surgical or therapeutic step.

It is emphasized again that the use of a patient interface producing rotationally asymmetrical pressure marks in the case of an ophthalmological device can be used independently of the system described here and the method described here. An ophthalmological device with a patient interface, the contact surface of which contains rotationally asymmetrical pressure markings which produce pressure marks in the cornea and/or sclera when the patient interface is fixed on the eye, as well as a method for marking the rotational position with which an eye was fixed on an ophthalmological device, wherein the patient interface just mentioned is used, therefore represent independent inventions. The same applies to the patient interface alone. The invention can be further developed in that the marking structures sit on the edge of a contact surface of the patient interface, which surface is placed on the eye. A further device, which can be an ophthalmological device for the further examination/treatment of the eye and which contains an imaging device for the detection of the position of the pressure marks, is advantageous for reading the pressure marks. The imaging device can comprise an illumination device for the glancing illumination of the cornea and/or sclera of the eye. Particularly preferably, in the further device, data input is effected which indicates the position of the pressure marks with respect to the first ophthalmological device which used the patient interface. Moreover, all of the features of the system and of the device for producing assistance information described here also come into consideration for the further development of the principle of marking the rotational position of an eye by means of pressure marks by means of a patient interface.

In addition to the replacement of the human lens of the eye by a plastic lens, additional cuts in the cornea can also be carried out within the framework of the operation which serve to correct corneal astigmatism. These cuts are usually carried out close to the limbus and consist of two cuts in the form of opposite segments of a circle (limbal relaxing incisions, LRI). These cuts can also be performed automatically with the laser cataract device.

A decisive parameter here is the angular orientation which should coincide with the cylinder orientation of the cornea surface. In addition, an annular illumination can be projected onto the surface of the cornea and the reflexes recorded with the camera inside the device. The axis of the ellipse in the image indicates the cylinder orientation of the surface of the cornea. The illumination can also consist of a series of light-emitting diodes which are arranged along a circle. The data of such LRIs can be included in the cutting geometry and thus in the position data set.

It is understood that the features mentioned above and those yet to be explained below can be used, not only in the stated combinations, but also in other combinations or alone, without departing from the scope of the present invention.

### BRIEF DESCRIPTION OF THE DRAWINGS

The invention is explained in further detail below by way of example with reference to the attached drawings which also disclose features essential to the invention. There are shown in:

FIG. 1 a schematic representation of a system for a cataract operation which provides information to assist a surgeon,

FIG. 2 a video image which was produced by the system of FIG. 1, and

FIG. 3 a top view of a contact surface of a patient interface which can be used with the system of FIG. 1.

### DETAILED DESCRIPTION

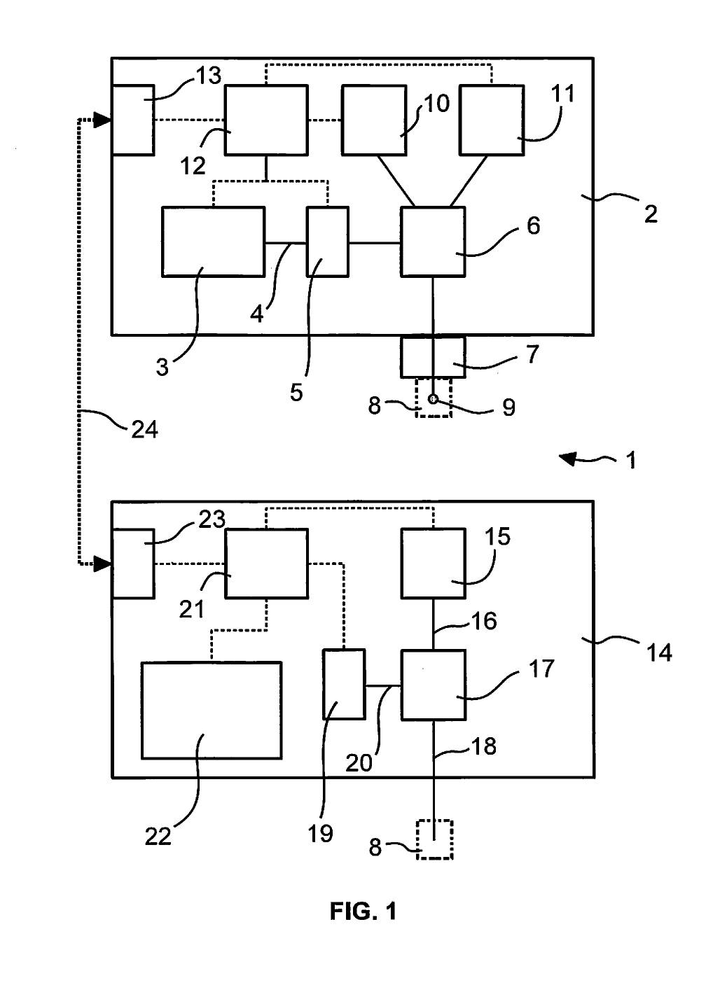

FIG. 1 shows a system 1 for a laser-based cataract operation. The system 1 comprises a cataract device 2 which comprises a laser 3 which emits, for example, pulsed laser radiation. A laser beam 4 provided by the laser 3 is deflected by means of a scanner 5 and the beam scanned in this way is focused into the eye 8 to be operated on via an optical system, not shown further, a beam splitter 6 and a patient interface 7. The pulsed laser radiation 4 brings about an optical perforation at a focal point in the eye 8 and thereby a cavitation 9 which divides tissue of the eye. By displacing the position of the focal point, a carpet of cavitations 9 forms which produces a cut surface in the eye 8. The position of the cut surface depends on the position of the cavitations 9 and thus on the displacement of the focal point, which the scanner 5 carries out.

The cataract device 2 comprises a video camera 10 which is coupled into the beam path to the eye 8 via the beam splitter 6 in such a way that the video camera 10 supplies an image of the eye 8 before, during and/or after the application of the laser beam 4. Furthermore, the cataract device 2 has a navigation system 11 which supplies structural information about the eye 8. The navigation system can, for example, be an OCT or a Scheimpflug camera which provides sectional representations of the eye.

The laser 3, the scanner 5, the video camera 10 and the navigation system 11 are connected to a control device 12, which can be designed, for example, as a suitably programmed computer, via control lines (drawn in with dotted lines in FIG. 1) which are not described further. The control device 12 controls the operation of the cataract device 2 and in particular the laser 3 and the scanner 5. The control of these two components is effected according to control data stored in the control device 12 which indicate the displacement of the focal point and thus the position of the cavitations 9. The control data are defined in a reference coordinate system which is related to the scanner 5. The patient interface 7 ensures that the eye 8 is located in a known and, in particular, unchangeable geometric position in relation to the cataract device 2. The surgeon's task is to ensure that a desired position is achieved when docking the patient interface 7 on the eye 8. Of course, the surgeon can also, optionally, predefine a change to the control data, i.e. deliberately change, e.g. displace, the cut surface in the eye. The navigation system 11 and/or the video camera 10 assists him in this.

The control data define, optionally in accordance with the influence and choice of the surgeon, a cutting geometry of at least one cut surface to be produced in the eye 8. Because of the reproducible operation of the scanner 5 and the laser 3, the control data which are used by the control device 12 are firmly linked to the cutting geometry of the cut/cuts produced or to be produced in the eye 8.

The control device 12 determines the position of reference structures in the eye 8 via the imaging device (video camera 10 and/or the navigation system 11). These can be the edge of the pupil, the edge of the limbus, sclera and/or iris structures. They have in common that they can be located on the eye 8 and enable the position of the eye 8 to be determined. Because the position of the imaging device in the cataract device 2 is known, the position of the imaging device in relation to the scanner 5 and thus to the reference coordinate system, in which the control data define the cut surface, is also known. From this given relationship, the control device 12 determines the position of the reference structures with respect to the coordinate reference system of the cataract device 2 and records it together with the control data in the form of a position data set and provides these to a data export module 13.

In addition to the cataract device 2, an operation microscope 14, which is also a component of the system 1, is used in the cataract operation. The system 1 is formed by the cataract device 2 and the operation microscope 14 which, however, are not used simultaneously but one after the other. The operation microscope 14 comprises a microscope 15 which images the eye 8 in a microscope beam path 16 which runs through a beam splitter 17 and an imaging beam path 18. Unlike in the cataract device 2, no patient interface is used here since the surgeon must have access to the eye 8 underneath the operation microscope 14 in order to be able to perform the surgical intervention. Furthermore, via the beam splitter 17, a camera 19 is coupled in which, independently of the microscope 15, provides a camera beam path 20, which the beam splitter 17 combines with the microscope beam path 16 to form the imaging beam path 18. The camera 19 thus supplies an image of the eye 8 and the microscope 15 supplies a greatly enlarged image of selectable sections of the eye 8.

The camera 19 and the microscope 15 are connected to a control unit 21 of the operation microscope 14 which reads the corresponding image data, in particular the image data which an image sensor of the microscope 15 and the camera 19 provide. The control unit 21 displays an image of the eye 8 on a display 22. This can be the image supplied by the camera 19 and/or the image supplied by the microscope 15. The display can also be designed as an eyepiece which displays the eye 8 by direct optical means or by electronic means via the microscope 15. In the case of a direct optical view, the microscope 15 has no image sensor and a reflecting device is present which reflects a display, which is appropriately actuated by the control unit 21, into the eyepiece of the microscope 15. The display 22 is then supplemented or replaced by the reflecting device.

It is important that the surgeon is provided with an image of the eye 8 via the display device (e.g. the display 22). The control unit 21 superimposes the cutting geometry of the cuts on this image. For this, it performs the following:

First of all, the control unit 21 imports the position data set, which was transferred from the data export module 13 via a data connection 24, into a data import module 23. In respect of the data connection 24, reference is made to the general part of the description.

From the position data set, the control unit imports the position of the reference structures with respect to the position of the cutting geometry. Optionally, the position data set can also contain details about the type of the reference structures.

The control unit then determines in the image of the eye 8, which was captured by the camera 19 and/or (in the case of an electronic design of the microscope 15) was supplied by the microscope 15, the position of the reference structures which the position data set indicates. Furthermore, the control unit determines an enlargement factor which is currently present for the image 22 to be displayed. It can result from the setting of the microscope 15 and/or the setting of the camera 19 and is appropriately derived by the control unit 21. The control unit then determines the position of the cutting geometry in the image displayed or to be displayed on the basis of the relative position of the cutting geometry in relation to the reference structures according to the position data set.

Finally, the control unit 21 superimposes the cutting geometry on the displayed image appropriately in terms of position and size.

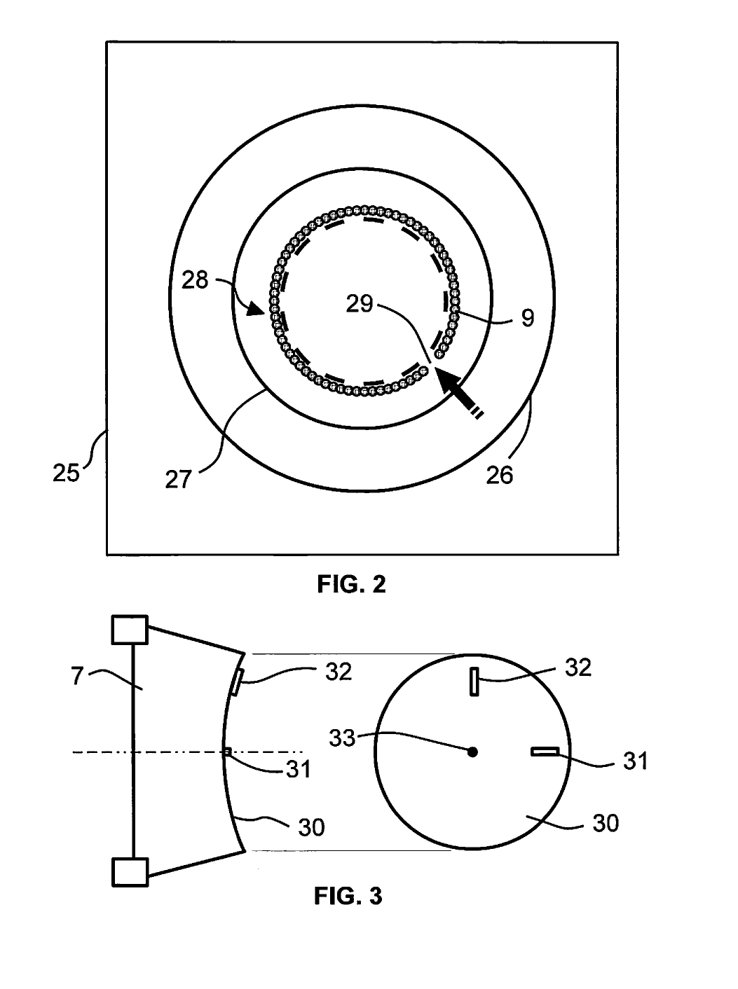

FIG. 2 shows, by way of example, a video image 25 as can be displayed on the display 22 or by reflection into an optical image of a microscope 15 provided with an optical view. The video image 25 allows the edge of the limbus 26 and the edge of the pupil 27 of the eye to be identified. Furthermore, a trail of successive cavitations 28 is represented which corresponds to a circular cutting line which is illustrated by means of a dashed circle. The cutting geometry of the cut appears in the top view as a line. In the video image 25, the trail 28 of cavitations 9 is not indicated, only the course of the cutting line drawn in with a dashed line in FIG. 2.

From the trail of successive cavitations 28 plotted by way of illustration in FIG. 2 it can be seen that there is a gap 29. Such a gap can, as explained in the general part of the description, be caused by a corneal fold. Such a gap or a point at which it can appear is marked in the video image 25. FIG. 2 shows, by way of example, a warning arrow which is superimposed at the point of a gap.

In an embodiment, the gap can be a detected gap. Then, the control device 12 evaluates the image supplied by the imaging device 10, 11 to the effect that after production of a cut surface, gaps in the cavitation trail are sought, identified and the locations thereof are recorded in the position data set. However, the gap can also be a gap which has formed unintentionally, for example when the cut surface has not yet been produced. A location at which a gap has formed unintentionally can, as explained in the general part of the description, be derived from the intersection of a corneal fold with a cut surface or cutting line to be produced. In this case, the control device 12 determines by evaluating the information from the imaging device (image of the video camera 10 and/or of the navigation system 11) are detected. To determine the intersection of the corneal fold with a cut surface/cutting line, the location of a potential gap can be identified and provided for recording in the position data set.

A reference structure, the position of which is recorded in the position data set, does not necessarily have to be a structure which occurs naturally in the eye. It can also be an artificially produced structure. FIG. 3 shows an embodiment example of a contact glass which can be used for producing such an artificial reference structure. In FIG. 3, a side view and a top view of a reference contact surface 30 of the contact glass are represented. Alternatively, instead of the contact glass, a (liquid, gas or solid) patient interface which has a reference structure or reference surface, can also be used. The contact surface 30 is placed on the front side of the eye, for example on the cornea and/or sclera. It brings the front surface of the eye into a particular shape and fixes the eye 8 with respect to the device which uses the patient interface, for example the cataract device 2. Fixing means with which the patient interface 7 is docked on the eye are not represented in FIG. 3. The fixing mechanism for attaching to the ophthalmological device in a defined rotational position is only indicated schematically.

Markings 31, 32 are formed on the contact surface 30 which are suitable for producing pressure marks in the front surface of the cornea. The markings 31, 32 can be projections or indentations. They lie asymmetrically in relation to an optical axis of the patient interface and of the device for which the patient interface is used. For the optical axis, the optical image centre 33 is plotted in the top view of FIG. 3. Because of the asymmetrical position of the markings 31 and 32, the pressure marks also lie rotationally asymmetrical in relation to the optical axis. From an evaluation of the pressure marks, which is particularly easily possible for example in a design with a glancing illumination, it can be easily established using a further ophthalmological device, for example the operation microscope 14, in which rotational position the eye 8 was fixed to the previously used ophthalmological device with the patient interface 7.

1. A system for producing assistance information within the framework of a laser-assisted cataract operation on an eye, wherein the system comprises:
a laser cataract device, which comprises
a laser beam source, which emits a laser beam and a laser beam scanning device for producing cuts in the cornea and/or capsular bag of the eye by scanning the laser beam in the cornea and/or capsular bag of the eye,
a control device for predefining a cutting geometry of the cuts by control data to control the laser beam scanning device,
an imaging device for imaging reference structures of the eye,
an output interface,
wherein the control device is designed to determine the position of the reference structures and to produce a position data set which contains the cutting geometry and the relative position thereof in relation to the reference structures and to output it on the interface,
an operation microscope, which comprises
a microscope and/or a camera for imaging the eye on an image sensor which supplies image data,
a display device for displaying an image of the eye according to the image data,
an input interface for receiving the position data set and
a control unit, which receives the position data set on the input interface, determines the position of the reference structures of the eye with respect to the image data and, on the display device, superimposes the cutting geometry contained in the position data set on the displayed image appropriately in terms of position and size with respect to the reference structures.

2. The system according to claim 1, wherein the control device identifies gaps in cuts by utilizing the imaging device and records the position thereof in the position data set and in that the control unit superimposes the locations of the gaps on the displayed image.

3. The system according to claim 1, wherein the control device identifies folds in the cornea of the eye by utilizing the imaging device and records the position thereof in the position data set and in that the control unit superimposes the locations of the folds on the displayed image.

4. The system according to claim 1, wherein the laser cataract device comprises a patient interface having a contact surface to be placed on the eye comprising pressure markings which are rotationally asymmetrical with respect to an optical axis and are suitable for producing pressure marks on the eye.

5. The system according to claim 1, wherein by means of the imaging device, the control device uses one or more of the following structures of the eye as reference structures, the position of which is determined: the edge of the pupil, the edge of the limbus, iris structures, sclera structures.

6. The system according to claim 1, wherein the control device records the cutting geometry in the position data set in the form of the control data.

7. The system according to claim 1, wherein the control device records the cutting geometry and the reference structures in the position data set with respect to a reference system of the laser beam scanning device.

8. A method for producing assistance information for a laser-assisted cataract operation which, produces cuts in the cornea and/or capsular bag of an eye by a laser cataract device controlled according to control data, wherein in the method the following steps are performed:
a) imaging the eye and determining a position of reference structures of the eye and determining a cutting geometry of the cuts produced or to be produced from the control data,
b) determining a relative position of the cutting geometry in relation to the reference structures and providing a position data set which contains the cutting geometry and the relative position thereof in relation to the reference structures,
c) transferring the position data set to an operation microscope,
d) producing and displaying an image of the eye with the operation microscope,
e) determining the position of the reference structures of the eye in the image produced and
f) with respect to the reference structures, superimposing on the displayed image the cutting geometry contained in the position data set appropriately in terms of position and size.

9. The method according to claim 8, wherein in step b), gaps are identified in cuts produced, the locations of which are recorded in the position data set and are superimposed on the displayed image.

10. The method according to claim 8, wherein in step b), folds are identified in the cornea of the eye the locations of which are recorded in the position data set and are superimposed on the displayed image.

11. The method according to claim 8, wherein when the laser cataract device is used, a contact glass or patient interface is used having a contact surface which is to be placed on the eye comprising pressure markings which are rotationally asymmetrical with respect to an optical axis and are suitable for producing pressure marks in the eye.

12. The method according to claim 8, wherein one or more of the following structures of the eye are used as reference structures, the position of which is determined: the edge of the pupil, the edge of the limbus, iris structures, sclera structures.

13. The method according to claim 8, wherein the cutting geometry is recorded in the position data set in the form of the control data.

14. The method according to claim 8, wherein the cutting geometry and the reference structures are recorded in the position data set with respect to a reference system of the laser cataract device.
